(19) United States
(12) Patent Application Publication
(10) Pub. No.: US 2010/0076294 A1
(43) Pub. Date: Mar. 25, 2010

(54) System and Method of Prepping Skin Prior to Electrode Application

(75) Inventors: Warren Copp-Howland, Chicopee, MA (US); Kathy Tremblay, Westfield, MA (US)

Correspondence Address: TYCO HEALTHCARE GROUP LP, 15 HAMPSHIRE STREET, MANSFIELD, MA, 02048, US

(73) Assignee: Tyco Healthcare Group LP, Mansfield, MA (US)

(21) Appl. No.: 12/237,768

(22) Filed: Sep. 25, 2008

(51) Int. Cl. A61B 5/04

(52) U.S. Cl. 600/391

(57) ABSTRACT

An electrode is provided that includes a conductive member defining a first side and a second side; a conductive composition disposed on the first side of the conductive member, wherein the conductive composition includes a therapeutically effective quantity of an exfoliant; and an electrical lead in electrical communication with the conductive member. The conductive composition is capable of reducing a level of an impedance of the skin of the subject.

### BACKGROUND

1. Technical Field

The present application relates to surgical/medical procedures and, more particularly, to electrodes and methods of manufacturing electrodes with conductive compositions containing exfoliants to aid in preparing the surface of the skin of a patient prior to an application of an electrode thereto.

2. Background of Related Art

Electrodes (e.g., diagnostic, therapeutic and/or monitoring type) are used to transmit electrical signals or currents between the body of a patient and external or remote equipment (e.g., diagnostic, therapeutic and/or monitoring type). Such electrodes typically include a conductive composition adherable to or otherwise contactable with, the skin of the patient, and a conductor, which is electrically connected to the conductive composition and to the external equipment.

The degree of effectiveness and/or responsiveness of the electrode is related to the characteristics of the under-lying skin of the patient. The skin naturally has a high impedance level. Typically, the higher the impedance, the more difficult it is for the electrical signal to penetrate the stratum corneum. The more difficult it is for the electrical signal to traverse the stratum corneum the more time is required for the electrical signal to be delivered and received and the quality of the electrical signal is reduced.

In practice, clinicians may prepare the skin by removing dead layers of stratum corneum prior to placing an electrode on the skin of a patient. Common procedures include the use of alcohol swabs, cleansing the skin with water, soaps, or detergents, and using an abrasive agent like pummus.

Accordingly, it is desirable to treat the skin of a patient in a manner which would reduce the impedance thereof, where improved signal acquisition and/or energy delivery may be achieved.

A further need exists for an electrode which reduces impedance.

Accordingly, a need exists for a system and/or method of treating the skin of a patient, prior to and/or during application of an electrode thereto, in order to reduce the level of impedance of the skin and thereby improve signal acquisition and/or energy delivery.

### SUMMARY

The present application relates to biomedical electrodes and methods of preparing the surface of the skin of a patient prior to an application of an electrode thereto.

According to an aspect of the present disclosure, an electrode for selective attachment to the skin of a subject, wherein the skin of the subject has an impedance, is provided. The electrode includes a conductive member defining a first side and a second side; a conductive composition disposed on the first side of the conductive member, wherein the conductive composition includes a therapeutically effective quantity of an exfoliant; and an electrical lead in electrical communication with the conductive member. In use, the conductive composition reduces a level of the impedance of the skin of the subject.

The conductive composition may be selectively adherable to the skin of a subject.

The exfoliant may be an alphahydroxy acid. The alphahydroxy acid may have a concentration of 1-100%. The conductive composition may include approximately 0.01-10% of alphahydroxy acid.

The electrode may include a backing member disposed on the second side of the conductive member. The electrode may include a release liner selectively, removably adhered to a surface of the conductive composition.

The conductive composition may be a hydrogel.

The electrode may include a reinforcement member supporting the conductive composition. The electrode may include a coating of silver provided on at least a portion of at least one of the first and second sides of the conductive member.

The electrical lead may be a pig-tail style lead, snap style lead or tab style lead.

According to another aspect of the present disclosure, a method of preparing the skin of a subject for the use of an electrode thereon, is provided. The method includes the steps of providing an electrode, wherein the electrode has a conductive member; a conductive composition disposed on a side of the conductive member, wherein the conductive composition includes a therapeutically effective quantity of an exfoliant; and an electrical lead in electrical communication with the conductive member. The method further comprises the step of adhering the electrode to the skin of a subject such that the conductive composition is in contact with the skin of the patient, wherein the exfoliant of the conductive composition reduces a level of impedance of the skin of the subject.

The exfoliant may be an alphahydroxy acid. The alphahydroxy acid may have a concentration of 1-100%. The conductive composition may include about 0.01-10% of alphahydroxy acid.

### DETAILED DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

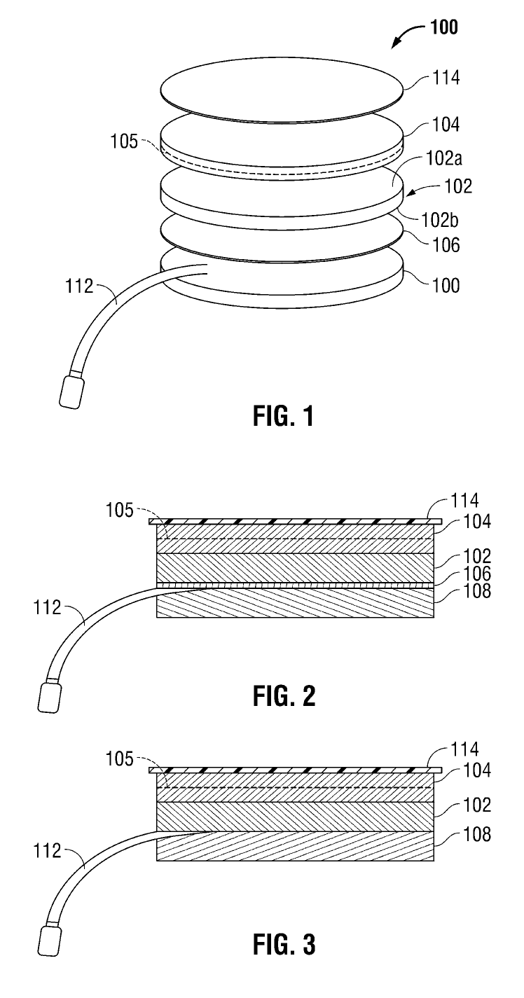

FIG. 1 is a schematic, perspective view of an exemplary leadwire style electrode of the present disclosure, shown with layers separated;

FIG. 2 is a cross-sectional view, of the electrode of FIG. 1;

FIG. 3 is a cross-sectional view, of an alternate leadwire style electrode, according to an embodiment of the present disclosure;

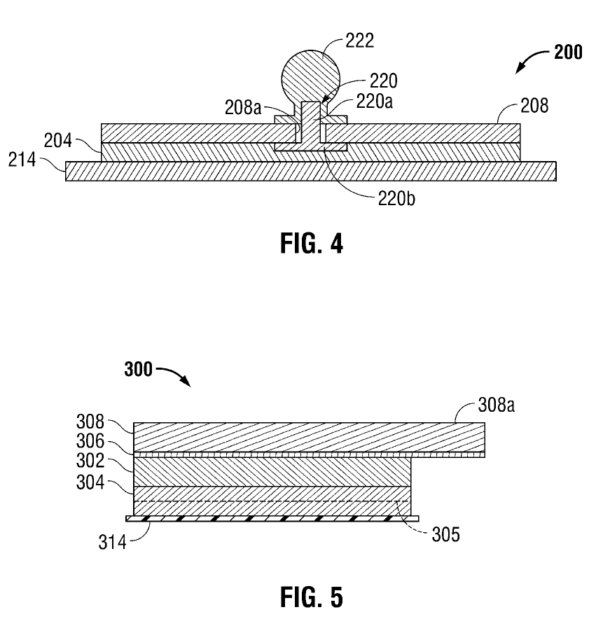

FIG. 4 is a cross-sectional view of an exemplary snap-type electrode of the present disclosure; and

FIG. 5 is a cross-sectional view, of a tab style electrode, according to an embodiment of the present disclosure.

### DETAILED DESCRIPTION OF EMBODIMENTS

Embodiments of the presently disclosed system and method will now be described in detail with reference to the drawing figures wherein like reference numerals identify similar or identical elements.

Referring initially to FIGS. 1 and 2, an electrode in accordance with an embodiment of the present disclosure is generally designated as electrode 100. Electrode 100 includes a conductive member 102 defining a first or skin side 102a relative to a subject and a second side 102b, opposite first side 102a. Conductive member 102 may be made from a conductive carbon, aluminum, tin or any other suitable conductive material. As an alternative, conductive member 102 may comprise a conductive plastic material. Conductive member 102 may include silver (Ag) or silver/silver-chloride (Ag/AgCl) material deposited on at least a portion of first side 102a or second side 102b. Either first side 102a or second side 102b may also have a coating of silver (Ag) or silver/silver-chloride (Ag/AgCl) composition or ink 106 on either first side 102a or second side 102b thereof.

Alternatively, as seen in FIG. 3, electrode 100 may be free of the coating of silver (Ag) or silver/silver-chloride (Ag/AgCl) composition or ink 106.

Turning back to FIGS. 1 and 2, electrode 100 further includes a conductive composition 104 disposed adjacent first side 102a of the conductive member 102 for application/adhesion to or contact with the skin of the subject. Conductive composition 104 may be made from, for example, but not limited to, Promeon RG-63B hydrogel (TycoHealthcare Group LP d/b/a Covidien). As seen in FIGS. 1 and 2, in some embodiments, conductive composition 104 may incorporate a reinforcement member. The reinforcement member may be a woven or non-woven cloth or gauze material (e.g., scrim) 105 embedded therewithin or supporting the structure of the hydrogel. The reinforcement member may be made from a conductive material. The conductive composition 104 may be any different commercially available conductive hydrogel. Conductive composition 104 is generally hydrophilic.

Conductive composition 104 has a therapeutically effective amount of an exfoliant incorporated therein. For example, conductive composition 104 may be provided with an alphahydroxy acid (AHA) incorporated therein. Conductive composition 104 may alternatively or further be provided with glycolic acid, citric acid, and/or boric acid incorporated therein. The exfoliant is added to conductive composition 104 in an amount sufficient to exfoliate the skin of the subject without or with minimal subsequent skin irritation to the patient. In an exemplary embodiment, the exfoliant has a concentration of about 0.01-5%.

By providing conductive composition 104 with a therapeutically effective quantity of an exfoliant, upon application of electrode 100 to the skin of the subject, at least some dead skin cells in the stratum corneum may be cleaned away or otherwise more easily penetrated. By reducing the thickness of the dead skin cells in the stratum corneum or by removing the dead skin cells in the stratum corneum, an impedance level of the skin may be reduced. As compared to the untreated skin of a subject, treated skin of the subject exhibits a reduced level of impedance, thereby reducing a relative time it takes for electrode 100 to acquire a signal for a sensing procedure and/or reducing a level of resistance encountered by electrode 100 for a stimulating procedure.

A first side release liner 114 is releasably secured to conductive composition 104. Release liner 114 can be made from a film or paper substrate having a release coating on one or both sides, such as, for example silicone. Release liner 114 protects and/or preserves conductive composition 104 (e.g., the hydrogel) and is removed prior to application on the skin of the subject. Release liner 114 may be applied to conductive composition 104 after use of electrode 100 to preserve the conductive composition 104 for subsequent use.

Release liner 114 may be a release paper or film of a waxed or coated plastic, such as a silicone coated polyethylene terephthalate film, which may be used to protect electrode 100 before application of the electrode to the skin of the subject.

In an embodiment, electrode 100 may further include a backing member 108 disposed adjacent second side 102b of conductive member 102. In certain embodiments, backing member 108 may overlie silver coating 106. Backing member 108 is fabricated from a non-conductive material such as a cloth, fabric, plastic material or the like.

Electrode 100 further includes an electrical lead or lead wire 112, e.g., a lead wire having a pig tail configuration that is in electrical communication with at least conductive member 102 and a power supply (not shown). Electrical communication extends from lead wire 112 through the conductive member 102 (and silver coating 106) and through conductive composition 104 to the subject.

In use, release liner 114 is removed from electrode 100. Electrode 100 is then applied to the skin of the subject, such that conductive composition 104 is adhered to the skin of the subject. Electrode 100 is then electrically connected to external medical equipment (not shown) by any connection means well known in the art, such as, for example, via lead wire 112. Other leads are well known in the art, including and not limited to snap style, tab style, rivet-post style, etc. Electrode 100 may, by way of example, be connected to a transcutaneous electrical nerve stimulation (TENS) unit by means known to one having skill in the art.

Turning now to FIG. 4, a cross-section of a snap-style electrode 200, in accordance with the present disclosure is shown. Electrode 200 includes a non-conductive backing member 208 having an opening 208a formed therein, and a conductive composition 204 supported thereon.

Electrode 200 includes an eyelet 220 having a stem 220a protruding through opening 208a, in a direction away from the skin of a subject, and a base 220b disposed or embedded in conductive composition 204 and being on the same side as the skin of the subject. Stem 208a of eyelet 208 is covered by a snap 222 which is securely connected thereto. Together snap 222 and eyelet 220 define at least part of a conductive pathway to provide an electrical connection between conductive composition 204 and the external medical equipment (not shown).

A release liner 214 may be provided to protect conductive composition 204 prior to use.

Similar to electrode 100, conductive composition 204 of electrode 200 includes a quantity of a therapeutically effective amount of an exfoliant therein. For example, conductive composition 204 may be provided with an alphahydroxy acid (AHA) incorporated therein. Conductive composition 204 may alternatively or further be provided with acetic, citric and/or boric acid incorporated therein. The exfoliant is added to conductive composition 204 in an amount sufficient to exfoliate the skin of the subject without or with minimal subsequent skin irritation to the patient. In an exemplary embodiment, the exfoliant has a concentration of about 0.01-5%.

Referring to FIG. 5, a cross-section of a tab-style electrode 300, in accordance with the present disclosure is shown. Electrode 300 includes a non-conductive backing member 308, and a conductive composition 304 supported thereon. Backing member 308 includes a portion 308a extending beyond a perimeter or edge of conductive member 202 thereby defining a tab. As seen in FIG. 5, silver coating 306 may extend onto the surface of tab portion 308a of backing member 308. A release liner 314 may be provided to protect conductive composition 304 prior to use.

Similar to electrodes 100 and 200, conductive composition 304 of electrode 300 includes a quantity of a therapeutically effective amount of an exfoliant therein. For example, conductive composition 304 may be provided with an alphahydroxy acid (AHA) incorporated therein. Conductive composition 304 may alternatively or further be provided with glycolic acid, citric acid, and/or boric acid incorporated therein. The exfoliant is added to conductive composition 304 in an amount sufficient to exfoliate the skin of the subject without or with minimal subsequent skin irritation to the patient. In an exemplary embodiment, the exfoliant has a concentration of about 0.01-5%.

It will be appreciated that various embodiments of the above-disclosure and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims. Unless specifically recited in a claim, steps or components of claims should not be implied or imported from the specification or any other claims as to any particular order, number, position, size, or material.

What is claimed is:

1. An electrode for selective attachment to the skin of a subject, wherein the skin of the subject has an impedance, the electrode comprising:
a conductive member defining a first side and a second side;
a conductive composition disposed on the first side of the conductive member, wherein the conductive composition includes a therapeutically effective quantity of an exfoliant; and
an electrical lead in electrical communication with the conductive member.

2. The electrode according to claim 1, wherein the exfoliant is an alphahydroxy acid.

3. The electrode according to claim 2, wherein the alphahydroxy acid has a concentration of 1-100%.

4. The electrode according to claim 2, wherein the conductive composition includes about 0.01-10% of alphahydroxy acid.

5. The electrode according to claim 1, further comprising a backing member disposed on the second side of the conductive member.

6. The electrode according to claim 1, further comprising a release liner selectively, removably adhered to a surface of the conductive composition.

7. The electrode according to claim 1, wherein the conductive composition is a hydrogel.

8. The electrode according to claim 1, further comprising a reinforcement member supporting the conductive composition.

9. The electrode according to claim 1, further comprising at least one of a coating of silver and silver-chloride on at least a portion of at least one of the first and second sides of the conductive member.

10. The electrode according to claim 1, wherein the electrical lead is one of a pig-tail style electrical lead, a snap style electrical lead and a tab style electrical lead.

11. A method of preparing the skin of a subject for the use of an electrode thereon, the method comprising the steps of:
providing an electrode, wherein the electrode includes:
a conductive member;
a conductive composition disposed on a side of the conductive member, wherein the conductive composition includes a therapeutically effective quantity of an exfoliant; and
an electrical lead in electrical communication with the conductive member;
adhering the electrode to the skin of a subject such that the conductive composition is in contact with the skin of the patient.

12. The method according to claim 11, wherein the exfoliant is an alphahydroxy acid.

13. The method according to claim 12, wherein the alphahydroxy acid has a concentration of 1-100%.

14. The method according to claim 12, wherein the conductive composition includes 0.01-10% of alphahydroxy acid.
